(19) United States
(12) Patent Application Publication
(10) Pub. No.: US 2015/0320656 A1
(43) Pub. Date: Nov. 12, 2015

(54) COSMETIC COMPOSITION OF HYDROPHOBIC SILICA AEROGEL PARTICLES AND AN ACRYLIC THICKENING POLYMER

(71) Applicant: L'OREAL, Paris (FR)

(72) Inventors: Samira KHENNICHE, Clichy (FR); Gregory PLOS, Paris (FR)

(73) Assignee: L'OREAL, Paris (FR)

(21) Appl. No.: 14/409,928

(22) PCT Filed: Jun. 20, 2013
(86) PCT No.: PCT/FR2013/051436

§ 371 Date: Dec. 19, 2014

Related U.S. Application Data
(60) Provisional application No. 61/728,265, filed on Nov. 20, 2012.

(30) Foreign Application Priority Data
Jun. 21, 2012 (FR) 1255855

(51) Int. Cl. A61K 8/25; A61Q 5/02; A61Q 5/00; A61K 8/02; A61K 8/81

(52) U.S. Cl. CPC A61K 8/25; A61K 8/0279; A61K 8/8147; A61Q 5/00; A61Q 5/02; A61Q 5/008; A61K 2800/412; A61K 2800/48; A61K 2800/612; A61K 2800/651

(57) ABSTRACT

The present invention relates to a cosmetic composition comprising hydrophobic silica aerogel particles and at least one particular acrylic thickening polymer.

The invention also relates to a cosmetic treatment method using said composition, especially a hair treatment method that makes it possible in particular to delay or even prevent regreasing of the hair and/or of the scalp.

The present invention relates to a cosmetic composition, in particular a hair composition, comprising the combination of hydrophobic silica aerogel particles and particular thickening polymers, and also to a method of using said composition.

Hair has a tendency to lose some of its qualities due to the action of factors such as natural regreasing, sweat, the removal of squamae, pollution, humidity and other factors. The visual appearance or the feel of the hair can thus be damaged. Regreasing, for example due to pollution, makes the hair lank, which then has a tendency to clump together. The hair may be difficult to style, in addition to having, for example, an unpleasant waxy feel or greasy sheen. In order to combat these inconveniences, it is possible to use detergent compositions, for example shampoos, in order to remove contaminants (sebum, sweat, pollution, etc.) or dandruff, and to loosen the hair. The hair is subsequently rinsed and then dried. The shampooing operations have to be repeated regularly, for example after a few days, or even after a few hours. However, shampoos, based on large amounts of surfactants, may cause discomfort such as stinging on the scalp or in the eyes.

Shampoo compositions or compositions for washing the skin may also combine, with the surfactants, sebum absorbers in order to make it possible to extend in time the perception of cleanliness of the hair or skin. However, it has been found that the effectiveness of these products is still insufficient with respect to the expectations of consumers. This is because they do not make it possible to significantly reduce the frequency of washing of the hair in particular, which frequency may differ depending on the country or even the region in question and which may range from one to two shampooing operations per day to one or two shampooing operations per week.

To date, no hygienic cosmetic composition, in particular hair composition, makes it possible to significantly slow down regreasing, in particular of the hair.

The objective of the present invention is to provide cosmetic compositions that overcome these disadvantages.

The compositions according to the invention make it possible to retain a perception of clean hair for a longer time than with a standard shampoo; by way of example, this perception of clean hair may be one week for people who normally wash their hair 2 to 3 times a week, and may be at least 3 days for people who normally wash their hair every day.

Furthermore, the compositions according to the invention make it possible to obtain cleansing performances at least identical to those of a standard shampoo, in particular a very good detergency power.

One subject of the invention is therefore a cosmetic composition comprising, in a cosmetically acceptable medium, hydrophobic silica aerogel particles and at least one particular acrylic thickening polymer, selected from a list.

It is been found that the use of the compositions according to the invention makes it possible to significantly reduce regreasing of the hair and/or of the scalp, and thus makes it possible to reduce the frequency of washing.

In addition, the composition according to the invention makes it possible to obtain good hair conditioning properties, especially in terms of softness, manageability, smoothing and disentangling, while having improved distribution and spreading over the hair.

In the present description, the expression “at least one” is equivalent to the expression “one or more” and can be replaced therewith.

In the present description, the expression “between” is equivalent to the expression “ranging from” and can be replaced therewith.

### Hydrophobic Silica Aerogel Particles

The composition according to the invention therefore comprises hydrophobic silica aerogel particles.

Aerogels are ultra-light porous materials, the first ones of which were made by Kristler in 1932.

They are generally synthesized via a sol-gel process in liquid medium and then dried by extraction of a supercritical fluid. The supercritical fluid most commonly used is supercritical CO₂. This type of drying makes it possible to avoid shrinkage of the pores and of the material. Other types of drying also make it possible to obtain porous materials from gel, namely for example (i) drying by cryodesiccation, which consists in solidifying the gel at low temperature and then in subliming off the solvent, and (ii) drying by evaporation. The materials thus obtained are then known, respectively, as cryogels and xerogels. The sol-gel process and the various drying operations are described in detail in Brinker C. J., and Scherer G. W., Sol-Gel Science, New York, Academic Press, 1990.

The term “hydrophobic silica” means any silica whose surface is treated with silylating agents, for example halogenated silanes such as alkylchlorosilanes, siloxanes, in particular dimethylsiloxanes such as hexamethyldisiloxane, or silazanes, so as to functionalize the OH groups with silyl groups Si—Rn, for example trimethylsilyl groups.

Preferably, the hydrophobic silica aerogel particles capable of being used in the present invention advantageously have a specific surface area per unit of mass (S_{M}) ranging from 500 to 1500 m²/g, preferably from 600 to 1200 m²/g and better still from 600 to 800 m²/g.

Preferably, the hydrophobic silica aerogel particles capable of being used in the present invention advantageously have an oil absorption capacity, measured at the wet point, ranging from 5 to 18 ml/g, preferably from 6 to 15 ml/g and better still from 8 to 12 ml/g of particles.

Preferably, the hydrophobic silica aerogel particles capable of being used in the present invention advantageously have a size, expressed as the mean diameter (D[0.5]), of less than 1500 μm, and preferably ranging from 1 to 30 μm, preferably from 5 to 25 μm, better still from 5 to 20 μm and even better still from 5 to 15 μm. Preferably, the hydrophobic silica aerogel particles capable of being used in the present invention advantageously have a tapped density p ranging from 0.04 g/cm³ to 0.10 g/cm³ and preferably from 0.05 g/cm³ to 0.08 g/cm³.

Preferably, the hydrophobic silica aerogel particles capable of being used in the present invention advantageously have a specific surface area per unit of volume (S_{V}) ranging from 5 to 60 m²/cm³, preferably from 10 to 50 m²/cm³ and better still from 15 to 40 m²/cm³.

According to one preferred embodiment, the hydrophobic silica aerogel particles according to the invention have a specific surface area per unit of mass (S_{M}) ranging from 500 to 1500 m²/g, preferably from 600 to 1200 m²/g and better still from 600 to 800 m²/g, and have a size, expressed as the mean diameter (D[0.5]), ranging from 1 to 30 μm and/or an oil absorption capacity, measured at the wet point, ranging from 5 to 18 ml/g, preferably from 6 to 15 ml/g and better still from 8 to 12 ml/g of particles.

According to another preferred embodiment, the hydrophobic silica aerogel particles used in the present invention have a specific surface area per unit of mass (S_{M}) ranging from 600 to 800 m²/g and a size, expressed as the volume mean diameter (D[0.5]), ranging from 5 to 20 μm and better still from 5 to 15 μm.

The specific surface area per unit of mass can be determined by the nitrogen absorption method, known as the BET (Brunauer-Emmett-Teller) method, described in The Journal of the American Chemical Society, vol. 60, page 309, February 1938, which corresponds to international standard ISO 5794/1 (appendix D). The BET specific surface area corresponds to the total specific surface area of the particles under consideration.

The absorption capacity measured at the wet point, denoted Wp, corresponds to the amount of oil which it is necessary to add to 100 g of particles in order to obtain a homogeneous paste. It is measured according to the “wet point” method or the method for determining the oil uptake of a powder according to the principle described in standard NF T 30-022. It corresponds to the amount of oil adsorbed onto the available surface of the powder and/or absorbed by the powder by measurement of the wet point, described below:

An amount m=2 g of powder is placed on a glass plate, and the oil (isononyl isononanoate) is then added dropwise. After addition of 4 to 5 drops of oil to the powder, mixing is carried out using a spatula, and addition of oil is continued until conglomerates of oil and powder have formed. From this point, the oil is added at the rate of one drop at a time and the mixture is subsequently triturated with the spatula. The addition of oil is stopped when a firm, smooth paste is obtained. This paste must be able to be spread on the glass plate without cracking or forming lumps. The volume Vs (expressed in ml) of oil used is then noted.

The oil uptake (oil absorption capacity) corresponds to the ratio Vs/m.

The sizes of the aerogel particles according to the invention may be measured by static light scattering using a commercial particle size analyser such as the MasterSizer 2000 machine from Malvern. The data are processed on the basis of the Mie scattering theory. This theory, which is exact for isotropic particles, makes it possible to determine, in the case of non-spherical particles, an “effective” particle diameter. This theory is especially described in the publication by Van de Hulst, H. C., “Light Scattering by Small Particles”, Chapters 9 and 10, Wiley, New York, 1957.

In the context of the present invention, the tapped density may be assessed according to the following protocol, known as the tapped density protocol: 40 g of powder are poured into a measuring cylinder and the cylinder is then placed on a Stay 2003 machine from Stampf Volumeter. The cylinder is then subjected to a series of 2500 tapping actions (this operation is repeated until the difference in volume between two consecutive tests is less than 2%); the final volume Vf of tapped powder is then measured directly on the cylinder. The tapped density is determined by the ratio: mass (m)/Vf, in this instance 40/Vf (Vf being expressed in cm³ and m in g).

The specific surface area per unit of volume is given by the relationship: S_{V}=S_{M}×ρ, where ρ is the tapped density, expressed in g/cm³, and S_{M} is the specific surface area per unit of mass, expressed in m²/g, as defined above.

The hydrophobic silica aerogel particles used according to the present invention are preferably silylated silica (INCI name: silica silylate) aerogel particles. The preparation of hydrophobic silica aerogel particles that have been surface-modified by silylation is described more fully in U.S. Pat. No. 7,470,725.

Use will be made in particular of aerogel particles of hydrophobic silica surface-modified with trimethylsilyl groups.

As hydrophobic silica aerogels that may be used in the invention, examples that may be mentioned include the aerogel sold under the name VM-2260 (INCI name: silica silylate), by the company Dow Corning, the particles of which have an average size of about 1000 microns and a specific surface area per unit of mass ranging from 600 to 800 m²/g.

Mention may also be made of the aerogels sold by the company Cabot under the references Aerogel TLD 201, Aerogel OGD 201 and Aerogel TLD 203, Enova Aerogel MT 1100 and Enova Aerogel MT 1200.

Use will be made more particularly of the aerogel sold under the name VM-2270 (INCI name: silica silylate), by the company Dow Corning, the particles of which have an average size ranging from 5 to 15 microns and a specific surface area per unit of mass ranging from 600 to 800 m²/g.

A mixture of hydrophobic silica aerogel particles may, of course, be used.

The compositions according to the invention may comprise the hydrophobic silica aerogel particles in an amount of between 0.01% and 20% by weight, preferably between 0.05% and 10% by weight and preferentially between 0.1% and 5% by weight, relative to the total weight of the composition.

### Thickening Polymers

The composition according to the invention also comprises at least one acrylic thickening polymer. A mixture of acrylic thickening polymers may, of course, be used.

The expression “acrylic polymer” is understood within the meaning of the present invention to mean a polymer that results from a polymerization reaction using one or more monomers of structure:

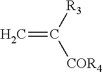

with R₃ denoting a hydrogen atom or a linear or branched C1-C4 alkyl radical, and R₄ denoting a hydrogen atom, a linear or branched C1-C4 alkyl radical, an NR₅R₆ radical, or a linear or branched C1-C30 alkoxy radical, said radicals being optionally substituted with one or more OH radicals and/or a quaternary ammonium radical,
R₅ and R₆ denoting, independently of one another, a hydrogen atom or an optionally oxyalkylenated linear or branched C1-C30 alkyl radical, optionally comprising a sulfonic group.

Preferably, R₃ denotes a hydrogen atom or a methyl radical.

The expression “thickening polymer” is understood, for the purposes of the present invention, to mean a polymer having, in solution or in dispersion containing 1% by weight of active material in water or in ethanol at 25° C., a viscosity greater than 0.2 poise at a shear rate of 1 s⁻¹. The viscosity is measured with a Haake RS600 viscometer from Thermo Electron. This viscometer is a controlled-stress viscometer with cone-plate geometry (for example having a diameter of 60 mm).

The acrylic thickening polymers capable of being used in the present invention are selected from:

(a) crosslinked acrylic acid homopolymers;
(b) crosslinked copolymers of (meth)acrylic acid and of a C1-C6 alkyl acrylate;
(c) nonionic homopolymers and copolymers containing ethylenically unsaturated monomers of ester and/or amide type;
(d) ammonium acrylate homopolymers or copolymers of ammonium acrylate and of acrylamide;
(e) optionally crosslinked (meth)acrylamido(C1-C4)alkylsulfonic acid homopolymers; and
(f) crosslinked methacryloyloxy(C1-C4)alkyltri(C1-C4)alkylammonium homopolymers and copolymers.
(a) Mention may be made, among the crosslinked acrylic acid homopolymers, of the products sold under the names Carbopol 980, 981, 954, 2984 and 5984 by the company Goodrich or the products sold under the names Synthalen M and Synthalen K by the company 3 VSA.
(b) Mention may be made, among the crosslinked copolymers of (meth)acrylic acid and of C1-C6 alkyl acrylate, of the product sold under the name Viscoatex 538C by the company Coatex, which is a crosslinked copolymer of methacrylic acid and of ethyl acrylate as an aqueous dispersion containing 38% active material, or the product sold under the name Aculyn 33 by the company Röhm & Haas, which is a crosslinked copolymer of acrylic acid and of ethyl acrylate as an aqueous dispersion containing 28% active material. Mention may be made more particularly of the crosslinked methacrylic acid/ethyl acrylate copolymer in the form of an aqueous 30% dispersion manufactured and sold under the name Carbopol Aqua SF-1 by the company Noveon.
(c) Mention may be made, among the nonionic homopolymers or copolymers containing ethylenically unsaturated monomers of ester and/or amide type, of the products sold under the names Cyanamer P250 by the company Cytec (poly-acrylamide); PMMA MBX-8C by the company US Cosmetics (methyl methacrylate/ethylene glycol dimethacrylate copolymer); Acryloid B66 by the company Röhm & Haas (butyl methacrylate/methyl methacrylate copolymer); and BPA 500 by the company Kobo (polymethyl methacrylate).
(d) Mention may be made, among the ammonium acrylate homopolymers, of the product sold under the name Microsap PAS 5193 by the company Hoechst. Mention may be made, among the copolymers of ammonium acrylate and of acrylamide, of the product sold under the name Bozepol C Nouveau or the product PAS 5193 sold by the company Hoechst (they are described in documents FR-2 416 723, US-2 798 053 and US-2 923 692).
(e) The (meth)acrylamido(C1-C4)alkylsulfonic acid homopolymers are preferably crosslinked. More particularly, they are partially or completely neutralized. These are water-soluble or water-swellable polymers. Mention may especially be made of polyacrylamidomethanesulfonic acid, polyacrylamidoethanesulfonic acid, poly-acrylamidopropanesulfonic acid, poly(2-acrylamido-2-methylpropanesulfonic acid), poly(2-methacrylamido-2-methylpropanesulfonic acid) and poly(2-acrylamido-n-butanesulfonic acid).

Crosslinked and partially or completely neutralized poly(2-acrylamido-2-methylpropanesulfonic acid) homopolymers are known, in particular from patent application DE-19625810.

They are generally characterized in that they comprise, randomly distributed:

a) from 90% to 99.9% by weight of units of formula (I) below:

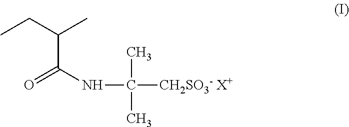

in which X⁺ denotes a cation or a mixture of cations, including H⁺,
b) from 0.01% to 10% by weight of at least one crosslinking unit containing at least two olefinic double bonds,
the proportions by weight being defined relative to the total weight of the polymer; X⁺ represents a cation or a mixture of cations chosen in particular from a proton, an alkali metal cation, a cation equivalent to that of an alkaline earth metal, or the ammonium ion.

Preferably, the crosslinked and neutralized poly(2-acrylamido-2-methyl propanesulfonic acid) comprises from 98% to 99.5% by weight of units of formula (I) and from 0.5% to 2% by weight of crosslinking units.

The crosslinking units having at least two olefinic double bonds are chosen, for example, from dipropylene glycol diallyl ether, polyglycol diallyl ethers, triethylene glycol divinyl ether, hydroquinone diallyl ether, tetraallyloxyethane or other poly-functional alcohol allyl or vinyl ethers, tetraethylene glycol diacrylate, triallylamine, trimethylolpropane diallyl ether, methylenebisacrylamide or divinylbenzene.

The crosslinking units having at least two olefinic double bonds are more particularly still chosen from those corresponding to the general formula (II) below:

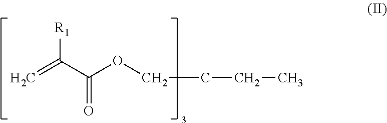

in which R₁ denotes a hydrogen atom or a C₁-C₄ alkyl and more particularly methyl (trimethylolpropane triacrylate).

The crosslinked and partially or completely neutralized poly(2-acrylamido-2-methylpropanesulfonic acids) are generally known under the names “Ammonium polyacrylamido-2-methylpropanesulfonate” or else “Ammonium polyacryldimethyl-tauramide” (INCI name).

A product that is particularly preferred according to the invention is the one sold by the company Clariant under the trade name Hostacerin AMPS; this is a crosslinked poly(2-acrylamido-2-methylpropanesulfonic acid) partially neutralized with aqueous ammonia.

(f) Mention may be made, among the crosslinked polymers of methacryloyloxy(C₁-C₄)alkyltri(C₁-C₄)alkylammonium salts, of the polymers obtained by homopolymerization of dimethylaminoethyl methacrylate quaternized with methyl chloride, or by copolymerization of acrylamide with dimethylaminoethyl methacrylate quaternized with methyl chloride, the homopolymerization or copolymerization being followed by crosslinking with an olefinically unsaturated compound, in particular methylenebisacrylamide. Use may more particularly be made of a crosslinked acrylamide/methacryloyloxyethyltrimethylammonium chloride copolymer (20/80 by weight) in the form of a dispersion containing 50% by weight of said copolymer in mineral oil. This dispersion is in particular sold under the name Salcare® SC 92 by the company Ciba. Use may also be made of a crosslinked homopolymer of methacryloyloxyethyltrimethylammonium chloride comprising approximately 50% by weight of the homopolymer in mineral oil or in a liquid ester. These dispersions are sold under the names Salcare® SC 95 and Salcare® SC 96 by the company Ciba.

Preferably, the acrylic thickening polymer or polymers are selected from crosslinked acrylic acid homopolymers.

The composition according to the invention comprises the acrylic thickening polymer or polymers in an amount preferably of between 0.01% and 2% by weight, in particular from 0.1% to 0.5% by weight, relative to the total weight of the composition.

### Other Ingredients

The cosmetic composition according to the invention generally comprises a cosmetically acceptable medium, i.e. a medium that is compatible with keratin materials such as the skin of the face or of the body, the lips, the hair, the eyelashes, the eyebrows and the nails.

The compositions according to the invention may be in any of the formulation forms conventionally used, and in particular in the form of an aqueous, alcoholic or aqueous-alcoholic, or oily solution or suspension; a solution or a dispersion of the lotion or serum type; an emulsion, in particular of liquid or semi-liquid consistency, of the O/W, W/O or multiple type; a suspension or emulsion of soft consistency of cream (O/W) or (W/O) type; an aqueous or anhydrous gel, or any other cosmetic form.

These compositions may be packaged in pump-action bottles or in aerosol containers, so as to apply the composition in vaporized (lacquer) form or in the form of a mousse. Such packaging forms are indicated, for example, when it is desired to obtain a spray or a mousse, for treating the hair. In these cases, the composition preferably comprises at least one propellant.

The composition according to the invention may be aqueous or anhydrous. It is preferably aqueous and then comprises water at a concentration preferably ranging from 5% to 98% by weight, especially from 20% to 95% by weight and better still from 50% to 90% by weight, relative to the total weight of the composition. The composition may also comprise one or more organic solvents that are liquid at 25° C. and 1 atm., and that are in particular water-soluble, such as C1-C7 alcohols; mention may especially be made of C1-C7 aliphatic or aromatic monoalcohols, and C3-C7 polyols and polyol ethers, which may thus be used alone or as a mixture with water. Advantageously, the organic solvent may be chosen from ethanol, isopropanol, benzyl alcohol and mixtures thereof.

The composition according to the invention may also comprise at least one standard cosmetic ingredient, other than the compounds of the invention, and in particular chosen from propellants; plant, mineral, animal or synthetic oils; solid fatty substances and in particular waxes, C8-C40 esters; C8-C40 acids; C8-C40 alcohols; nonionic surfactants, cationic surfactants, anionic surfactants, amphoteric surfactants, zwitterionic surfactants; sunscreens; moisturizers; antidandruff agents; antioxidants; chelating agents; reducing agents; oxidation bases, couplers, oxidizing agents, direct dyes; hair straightening agents such as thiols and alkali metal hydroxides; nacreous agents and opacifiers; plasticizers or coalescers; hydroxy acids; pigments; fillers; silicones and in particular polydimethylsiloxanes (PDMSs); conditioning or styling polymers; fragrances; basifying agents or acidifying agents; silanes; crosslinking agents such as polyphenols, aldehydes and DHA. The composition can, of course, comprise several cosmetic ingredients appearing in the above list.

Depending on their nature and the purpose of the composition, the standard cosmetic ingredients can be present in standard amounts which can be easily determined by those skilled in the art and which can be, generally, for each ingredient, between 0.01% and 80% by weight.

The oils may be preferably present in a proportion of from 0.01% to 80% by weight, especially from 0.02% to 40% by weight or even from 0.5% to 20% by weight, relative to the total weight of the composition. They may be carbon-based. Mention may especially be made of hydrogenated or non-hydrogenated plant, animal or mineral oils, saturated or unsaturated, linear or branched, cyclic or aliphatic, hydrocarbon-based synthetic oils, for instance poly(alpha-olefin)s, in particular polydecenes and polyisobutenes; liquid fatty alcohols such as isostearyl alcohol, octyldodecanol, 2-butyloctanol, 2-hexyldecanol, 2-undecylpentadecanol, oleyl alcohol or linoleyl alcohol; liquid esters such as isopropyl myristate or isopropyl palmitate. Mention may also be made of water-soluble or water-insoluble, organomodified or non-organomodified, volatile or non-volatile silicone oils; fluoro or perfluoro oils; and also mixtures thereof.

The composition may also comprise one or more solid fatty substances, and in particular one or more fatty alcohols, fatty esters and/or fatty acids, other than the above oils, having 8 to 40 carbon atoms; these solid fatty substances may preferably be present in a proportion of from 0.01% to 30% by weight, especially 0.1% to 20% by weight relative to the total weight of the composition. Mention may especially be made of C12-C32, especially C12-C26, linear-chain fatty alcohols, and in particular cetyl alcohol, stearyl alcohol, cetylstearyl alcohol and behenyl alcohol. Mention may also be made of C16-C40 linear-chain or branched-chain fatty acids, and especially 18-methyleicosanoic acid, coconut oil or hydrogenated coconut oil acids; stearic acid, lauric acid, palmitic acid and oleic acid, behenic acid, and mixtures thereof. Preferably, the fatty acids are not salified. Mention may also be made of the linear-chain fatty esters, comprising in total between 8 and 40 carbon atoms, such as myristyl, cetyl or stearyl myristates, palmitates and stearates, alone or as a mixture.

A person skilled in the art will take care to choose the ingredients included in the composition, and also the amounts thereof, such that they do not harm the properties of the compositions of the present invention.

The pH of the composition, if it is aqueous, may be acidic, neutral or alkaline. Preferably, the composition has a pH of between 2 and 11, in particular from 3 to 9, or even 3 to 7.

The cosmetic composition according to the invention may be in the form of a product for caring for, cleansing and/or making up bodily or facial skin, the lips, the eyebrows, the eyelashes, the nails and the hair, an antisun or self-tanning product, a body hygiene product, or a haircare product, especially for caring for, cleansing, styling, shaping or coloring the hair.

The composition especially finds a particularly advantageous use in the field of haircare, especially for holding the hairstyle or shaping the hair, or for the care, cosmetic treatment or cleansing of the hair. The haircare compositions are preferably shampoos, hair conditioners, styling or care gels, care lotions or creams, conditioners, masks, serums, hairsetting lotions, blow-drying lotions, hair styling and fixing compositions such as lacquers or sprays; hair restructuring lotions; lotions or gels for preventing hair loss, antiparasitic shampoos, antidandruff lotions or shampoos, and anti-seborrhea treatment shampoos. The lotions may be packaged in various forms, especially in vaporizers, in pump-action bottles or in aerosol containers so as to apply the composition in vaporized form or in the form of a mousse.

The composition according to invention finds a particularly advantageous use for caring for, treating and/or cleansing the hair and/or scalp, for example as a shampoo, as conditioner, as a shampoo and/or conditioner post-treatment, or else between two shampooing operations.

It is thus possible, for example, to apply the composition according to the invention to the scalp and/or the hair, directly after shampooing, on wet hair or on dry hair, with or without a leave-on time, with or without heat, said application optionally being followed by rinsing and drying, either at room temperature or with a hairdryer or else with a hair straightener (250° C.) for example.

It is also possible to apply the composition according to the invention to the scalp and/or the hair, directly after conditioning, on wet hair or on dry hair, with or without a leave-on time, with or without heat, said application optionally being followed by rinsing and drying, either at room temperature or with a hairdryer or else with a hair straightener (250° C.) for example.

It is also possible to apply the composition according to the invention to the scalp and/or the hair between two shampooing operations, on wet hair or dry hair.

The cosmetic composition may or may not be rinsed out after having been applied to the keratin materials (hair and/or scalp).

Another subject of the invention is a cosmetic treatment method, especially for making up, caring for, cleansing, coloring or shaping keratin materials, especially bodily or facial skin, the lips, the nails, the hair and/or the eyelashes, comprising the application to said materials of a cosmetic composition according to the invention.

It is in particular a hair treatment method for the care, cosmetic treatment and/or cleansing of the hair and/or of the scalp, and in particular that makes it possible to delay or even prevent regreasing of the hair and/or of the scalp, and thus to space out the shampooing operations.

The invention is illustrated in greater detail in the examples that follow.

### EXAMPLE 1

The following hair care composition is prepared (% by weight):

| | Composition |
|---|---|
| SILICA SILYLATE (INCI name) | 0.5% |
| Acrylic polymer (Carbopol 980) | 0.3% |
| Water | qs 100% |

The SILICA SILYLATE used is the product sold under the name DOW CORNING VM-2270 AEROGEL FINE PARTICLES by Dow Corning.

The composition is in the form of a dispersion having a pH equal to 3.5 and is easy to apply to the hair and the scalp; it makes it possible to reduce the regreasing of the hair.

1.-14. (canceled)

15. A cosmetic composition comprising hydrophobic silica aerogel particles and at least one acrylic thickening polymer chosen from:
(a) crosslinked acrylic acid homopolymers;
(b) crosslinked copolymers of (meth)acrylic acid and of a C1-C6 alkyl acrylate;
(c) nonionic homopolymers and copolymers containing ethylenically unsaturated monomers of ester and/or amide type;
(d) ammonium acrylate homopolymers or copolymers of ammonium acrylate and of acrylamide;
(e) optionally crosslinked (meth)acrylamido(C1-C4)alkylsulfonic acid homopolymers; and/or
(f) crosslinked methacryloyloxy(C1-C4)alkyltri(C1-C4)alkylammonium homopolymers and copolymers.

16. The composition of claim 15, wherein the hydrophobic silica aerogel particles have a specific surface area per unit of mass (S_{M}) ranging from about 500 m²/g to about 1500 m²/g.

17. The composition of claim 16, wherein the hydrophobic silica aerogel particles have a specific surface area per unit of mass (S_{M}) ranging from about 600 m²/g to about 800 m²/g.

18. The composition of claim 15, wherein the hydrophobic silica aerogel particles have an oil absorption capacity, measured at the wet point, ranging from about 5 mL/g to about 18 mL/g.

19. The composition of claim 18, wherein the hydrophobic silica aerogel particles have an oil absorption capacity, measured at the wet point, ranging from about 8 mL/g to about 12 mL/g of particles.

20. The composition of claim 15, wherein the hydrophobic silica aerogel particles have a size, expressed as the mean diameter (D[0.5]), of less than about 1500 μm.

21. The composition of claim 20, wherein the hydrophobic silica aerogel particles have a size, expressed as the mean diameter (D[0.5]), ranging from about 5 μm to about 15 μm.

22. The composition of claim 15, wherein the hydrophobic silica aerogel particles have a tapped density p ranging from about 0.04 g/cm³ to about 0.10 g/cm³.

23. The composition of claim 22, wherein the hydrophobic silica aerogel particles have a tapped density p ranging from about 0.05 g/cm³ to 0.08 g/cm³.

24. The composition of claim 15, wherein the hydrophobic silica aerogel particles have a specific surface area per unit of volume (S_{V}) ranging from about 5 m²/cm³ to about 60 m²/cm³.

25. The composition of claim 24, wherein the hydrophobic silica aerogel particles have a specific surface area per unit of volume (S_{V}) ranging from about 15 m²/cm³ to about 40 m²/cm³.

26. The composition of claim 15, wherein the hydrophobic silica aerogel particles are silylated silica aerogel particles.

27. The composition of claim 26, wherein the hydrophobic silica aerogel particles are aerogel particles of hydrophobic silica surface-modified with trimethylsilyl groups.

28. The composition of claim 15, wherein the hydrophobic silica aerogel particles are present in an amount ranging from about 0.01% to about 20% by weight, relative to the total weight of the composition.

29. The composition of claim 15, wherein the hydrophobic silica aerogel particles are present in an amount ranging from about 0.1% to about 5% by weight, relative to the total weight of the composition.

30. The composition of claim 15, wherein the at least one acrylic thickening polymer is chosen from crosslinked acrylic acid homopolymers.

31. The composition of claim 15, wherein the at least one acrylic thickening polymer is present in an amount ranging from about 0.01% to about 2% by weight, relative to the total weight of the composition.

32. The composition claim 15, comprising water at a concentration ranging from about 5% to about 98% by weight, relative to the total weight of the composition.

33. A cosmetic treatment composition comprising hydrophobic silica aerogel particles and at least one acrylic thickening polymer chosen from:
(a) crosslinked acrylic acid homopolymers;
(b) crosslinked copolymers of (meth)acrylic acid and of a C1-C6 alkyl acrylate;
(c) nonionic homopolymers and copolymers containing ethylenically unsaturated monomers of ester and/or amide type;
(d) ammonium acrylate homopolymers or copolymers of ammonium acrylate and of acrylamide;
(e) optionally crosslinked (meth)acrylamido(C1-C4)alkylsulfonic acid homopolymers; and/or
(f) crosslinked methacryloyloxy(C1-C4)alkyltri(C1-C4)alkylammonium homopolymers and copolymers,
wherein the composition is in the form of a hair product for the care, cosmetic treatment or cleansing of at least one of the hair and the scalp.

34. A method for the cosmetic treatment of keratin materials, comprising: applying to said keratin materials a composition comprising hydrophobic silica aerogel particles and at least one acrylic thickening polymer chosen from:
(a) crosslinked acrylic acid homopolymers;
(b) crosslinked copolymers of (meth)acrylic acid and of a C1-C6 alkyl acrylate;
(c) nonionic homopolymers and copolymers containing ethylenically unsaturated monomers of ester and/or amide type;
(d) ammonium acrylate homopolymers or copolymers of ammonium acrylate and of acrylamide;
(e) optionally crosslinked (meth)acrylamido(C1-C4)alkylsulfonic acid homopolymers; and/or
(f) crosslinked methacryloyloxy(C1-C4)alkyltri(C1-C4)alkylammonium homopolymers and copolymers.
